(19) United States
(12) Patent Application Publication
(10) Pub. No.: US 2010/0137685 A1
(43) Pub. Date: Jun. 3, 2010

(54) DEVIATED VIEWING RIGID VIDEOENDOSCOPE WITH ADJUSTABLE FOCUSING

(75) Inventor: Jean Rovegno, La Ciotat (FR)

Correspondence Address: SEED INTELLECTUAL PROPERTY LAW GROUP PLLC, 701 FIFTH AVE, SUITE 5400, SEATTLE, WA, 98104, US

(73) Assignee: TOKENDO, La Ciotat (FR)

(21) Appl. No.: 12/628,865

(22) Filed: Dec. 1, 2009

(30) Foreign Application Priority Data
Dec. 2, 2008 (FR) 0806750

(51) Int. Cl. A61B 1/04

(52) U.S. Cl. 600/109

(57) ABSTRACT

The disclosure relates to a deviated viewing rigid videoendoscopic probe with adjustable focusing, comprising a rigid inspection tube and a control handle attached to the inspection tube, the inspection tube having a longitudinal axis and a distal part comprising an image capture device having a viewing axis different from the longitudinal axis, the control handle comprising a rotation control device for making the viewing axis turn around the longitudinal axis, and a focusing control device of the image capture device for longitudinally moving in the inspection tube a part of the image capture device, the rotation control device and the focusing control device being respectively configured to make the viewing axis turn and focus the image capture device, independently from one another.

### BACKGROUND

1. Technical Field

The present disclosure relates to deviated viewing rigid videoendoscopes. The present disclosure applies particularly, but not exclusively, to endoscopes and videoendoscopes for medical purpose, and more particularly to laparoscopy.

2. Description of the Related Art

The term “endoscope” or “fiberscope” usually refers to an endoscopic probe comprising a distal end susceptible of being introduced into a dark cavity, so as to observe inside the cavity through an eyepiece. To that end, an endoscope comprises an optical device and a lighting device.

The optical device comprises a distal objective, a device for optically carrying the image supplied by the distal objective, and an eyepiece allowing the user to observe the image transmitted by the carrying device. The objective is housed in the distal end of an inspection tube behind a distal window. The optical carrying device housed in the inspection tube may be rigid and comprise a series of lenses, or soft and comprise a beam of ordered optical fibers. A focusing control ring on the endoscope handle may allow the image sharpness to be adjusted by moving an eyepiece lens along the optical axis thereof. It is to be noted that optical endoscopes for medical purpose are usually not provided with such a sharpness adjustment.

The lighting device comprises a continuous beam of optical fibers successively passing from the distal end of the probe, through the inspection tube, the control handle, and in the duct of an umbilical tube. The proximal end of the beam of fibers comprises a proximal endpiece to connect to a light generator. The distal end of the beam of fibers lights up the field of the objective through a lighting window. The lighting axis of the lighting window is in these conditions parallel to the optical axis of the distal window.

An endoscope may have an axial or deviated view. In an axial viewing endoscope, the optical axis of the distal window is merged with the mechanical axis of the inspection tube. The lighting window generally has the shape of a crown arranged around the distal window. In a deviated viewing endoscope, the optical axis of the distal window forms an angle with the mechanical axis of the inspection tube. The view is called “forward” if this angle is inferior to 90°, “lateral” if it is equal to 90° and “retrograde” if it is superior to 90°. In every instance, the optical device of a deviated viewing endoscope comprises a deviating prism located between the distal window and the distal objective of the endoscope. The lighting window at the distal end of the lighting device is usually arranged between the distal window and the distal end of the inspection tube.

The operating difficulties proper to conventional deviated viewing endoscopes lie in the panoramic exploration of the interior of a cavity. Such an exploration implies in fact that the user makes the endoscope turn by 360° around the mechanical axis thereof. This operation is rendered difficult by the presence of the lighting cable attached to a lighting base of the endoscope.

These operating difficulties are at the origin of the development of deviated viewing rotating endoscopes referred to according to manufacturers as “Rotascope” (HENKE-SASS WOLF), “Rotating shell endoscope” (EFER), “Boroscope with rotating light connector” (KARL STORZ), “Technoscope with rotating light connector” (RICHARD WOLF) or “Orbital scanning borescope” (OLYMPUS). All these endoscopes comprise a deviated viewing inspection tube which proximal end turns into a handle provided with a ring controlling the rotation of the inspection tube. These endoscopes also comprise a lateral base for connecting the lighting cable, a focusing adjustment ring, and an auxiliary lens for proximal vision. This type of architecture allows the user to rotate the endoscopic probe around its axis, without modifying the position of the lighting cable connected to the lighting base of the endoscope.

Deviated viewing rotating endoscopes have been described in the patents GB 2 280 514 (or U.S. Pat. No. 5,540,650 or EP 0 636 915), FR 2 762 102, FR 2 783 610 (or U.S. Pat. No. 6,346,076 or GB 2 342 462 or DE 1994 2152), FR 2 783 937, and FR 2 832 516, (or U.S. Pat. No. 6,817,976 or DE 6020 3242).

The term “videoendoscope” generally refers to an endoscopy system allowing the image of a target located in a dark cavity to be observed on a video screen. A videoendoscopic system comprises a camera associated to a conventional endoscope or a videoendoscopic probe. The camera or the videoendoscopic probe is associated to additional operation devices such as a power supply, a light generator and a visualization video monitor.

A videoendoscopic probe generally comprises:

a soft or rigid inspection tube comprising a distal endpiece,

a control handle attached to the proximal end of the inspection tube,

an umbilical tube which distal end is attached to the control handle,

a lighting device, and

a video processor.

The distal endpiece houses an optoelectronic device of small dimensions comprising in particular an objective and an image sensor, for example of the type “interline transfer tree-CCD sensor”. The image sensor comprises a photosensitive surface onto which forms the image provided by an objective. The proximal end of the umbilical tube comprises a fiber light connector and a multi-pin electrical connector allowing the probe to be connected to auxiliary operation devices which are functionally associated thereto.

The lighting device generally comprises a beam of lighting fibers successively housed in the umbilical tube, the control handle, and the inspection tube. The distal end of the beam of lighting fibers is integrated into the distal endpiece to light the target when its proximal end, provided with the light connector, is connected to a light generator.

The video processor which may be integrated into the control handle, is configured to transform into a useful video signal the electrical signal supplied by the distal image sensor. The video processor is linked to the image sensor by a multicore electric cable housed in the inspection tube. The synchronization of the image sensor with the video processor is originally adjusted according to the length and characteristics of the multicore cable. A control keypad, generally integrated into an operation box connected to the probe, allows the user to choose the operating parameters of the video processor.

The rigid videoendoscopic probes used in some medical applications, and in particular in interventional laparoscopy, may be of various types. In a first type, the probe is provided with a tip deflection which may be directed to two perpendicular planes and an axial viewing distal objective. In a second type, the probe is provided with an axial viewing distal objective and, generally, a focusing adjustment device. Such an adjustment device, which is described in particular in the patent FR 2 737 650, allows the distance between the image sensor and the proximal face of the distal objective to be longitudinally adjusted. The provision of such an adjustment device makes it possible to have an objective with small depth of field and therefore big aperture allowing a better global sensitivity to be obtained. In a third type, the probe is provided with a forward viewing distal objective which optical deviation is equal to 30° or 45° models.

### BRIEF SUMMARY

It is desirable to integrate into a rigid deviated viewing videoendoscopic probe, a focusing adjustment device and an orbital exploration device allowing the optical viewing axis to rotate on substantially 360° or more around the longitudinal mechanical axis of the probe.

In one embodiment, a deviated viewing rigid videoendoscopic probe with adjustable focusing is provided, comprising a rigid inspection tube and a control handle attached to the inspection tube, the inspection tube having a longitudinal axis and a distal part comprising an image capture device having a viewing axis different from the longitudinal axis. According to one embodiment, the control handle comprises a rotation control device for making the viewing axis turn around the longitudinal axis, and a focusing control device of the image capture device for longitudinally moving in the inspection tube a part of the image capture device, the rotation control device and the focusing control device being respectively configured to make the viewing axis turn and focus the image capture device independently from one another.

According to one embodiment, the image capture device comprises an image sensor comprising a photosensitive surface on which forms an image supplied by an objective and a deviating optical element associated to the objective to laterally deviate the optical axis of the objective according to the viewing axis, the focusing control device axially moving the image sensor in relation to the objective.

According to one embodiment, the focusing control device comprises an axially mobile tube, comprising a distal end attached to the image sensor and which axial movement is controlled by the focusing control device.

According to one embodiment, the focusing control device comprises a control ring mounted mobile in rotation on the control handle, and mechanically coupled to a freely rotating ring so that a rotation of the control ring causes an axial translation of the freely rotating ring, the freely rotating ring being mechanically coupled in translation, but not in rotation, to a central part mechanically coupled to the image capture device.

According to one embodiment, the focusing control device comprises a device for adjusting the axial clearance comprising an axial spring for returning the set consisting of the freely rotating ring and the central part toward an extreme position.

According to one embodiment, the rotation control device is configured to make the set consisting of the inspection tube and the image capture device turn.

According to one embodiment, the rotation control device comprises a control ring mounted mobile in rotation around the distal end of the control handle, and attached to the inspection tube and a cylindrical tube mobile in rotation but not in translation in the control handle, the cylindrical tube being mechanically coupled in rotation, but not in translation to a central part mechanically coupled to the image capture device.

According to one embodiment, the rotation control device comprises a stop attached to the control handle limiting in both directions the rotation of the viewing axis.

According to one embodiment, the rotation control device comprises a mobile stop device configured to extend the maximum rotation angle of the viewing angle to a value superior to 360°.

According to one embodiment, the rotation control device comprises a control ring attached to the inspection tube and a cylindrical tube mobile in rotation in the control handle, the mobile stop device comprising a freely rotating ring mechanically coupled in rotation to the cylindrical tube by a finger moving in an annular slot made in the freely rotating ring, the freely rotating ring comprising a finger cooperating with a stop attached to the control handle.

According to one embodiment, the probe comprises a beam of lighting fibers axially passing through the control handle and the inspection tube up to a lighting window arranged at the distal end of the inspection tube.

According to one embodiment, the beam of lighting fibers passes through the inspection tube at the exterior of a focusing control tube, axially mobile in the inspection tube.

According to one embodiment, the control handle comprises a chamber housing loops of the beam of lighting fibers and loops of a multicore cable linked to the image capture device.

According to one embodiment, the probe comprises a video processor housed in a chamber of the control handle and linked to the image capture device by a multicore cable axially passing through the inspection tube.

According to one embodiment, the multicore cable axially passes through the inspection tube in an axially mobile central tube in the inspection tube.

### BRIEF DESCRIPTION OF THE SEVERAL VIEWS OF THE DRAWINGS

Embodiments of the disclosure will be described hereinafter, in relation with, but not limited to the appended figures wherein:

FIG. 1 is a perspective view of a videoendoscope according to one embodiment,

FIG. 2 is a longitudinal sectional view of a videoendoscopic probe according to one embodiment,

FIG. 3 is a perspective and partial sectional view of rotation and focusing control mechanical devices, implemented in the videoendoscopic probe shown in FIG. 2,

FIG. 3A is a perspective view of a part of the mechanical devices shown in FIG. 2 or 3,

FIG. 4 is a transverse sectional view of the control handle.

### DETAILED DESCRIPTION

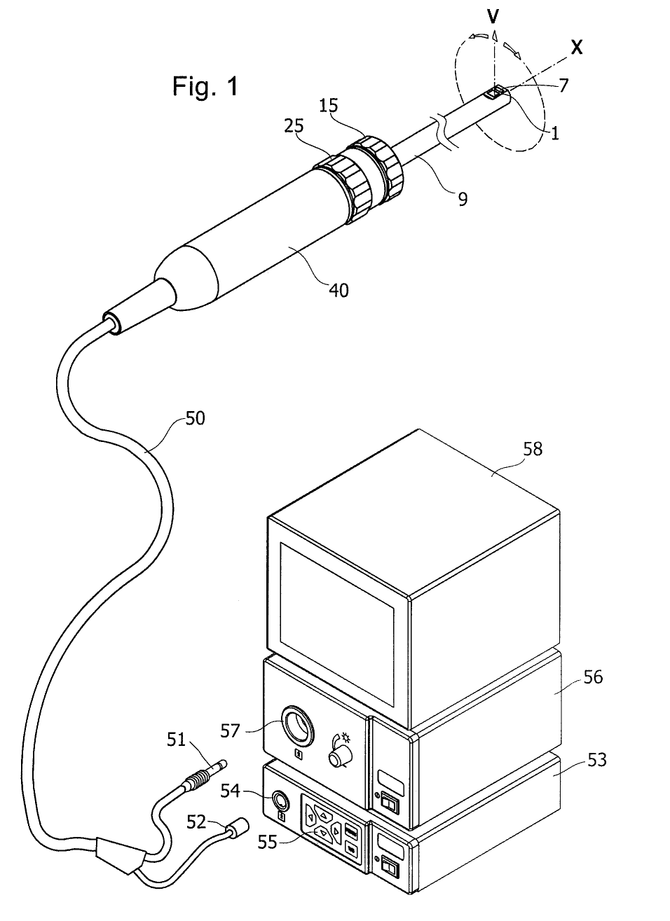

FIG. 1 shows the general architecture of a deviated viewing rigid videoendoscope, according to one embodiment. In FIG. 1, the videoendoscope comprises:

a probe comprising a rigid inspection tube 9 and a cylindrical control handle 40 attached to the tube 9,

an umbilical cable 50 attached to the handle 40,

an operation box 53 and a light generator 56, which may be connected to the cable 50, and

a video monitor 58 linked to the operation box 53.

The inspection tube 9 has a distal part comprising a lateral optical window 1 and a lighting window 7. The optical window has an optical axis (perpendicular to the optical window) defining the viewing axis V of the probe, which differs from the longitudinal axis X of the inspection tube 9. The handle 40 has a distal end attached to the proximal end of the inspection tube 9. The proximal end of the handle is attached to the distal end of the umbilical cable 50. The handle is encircled by a focusing control ring 25 and a rotation control distal ring 15 controlling the rotation of the inspection tube 9 around its axis X. The umbilical cable 50 has a proximal end provided with a multi-pin electrical connector 52 and a fiber light connector 51.

The operation box 53 comprises a control panel 55 allowing the video parameters of the videoendoscope to be adjusted and a multi-pin connection base 54 intended for receiving the connector 52 of the umbilical cable. The light generator 56 comprises a connection base 57 provided for receiving the light connector 51. The video monitor 58 allows the image of the target, supplied by the operation box 53, to be visualized in front of the window 1.

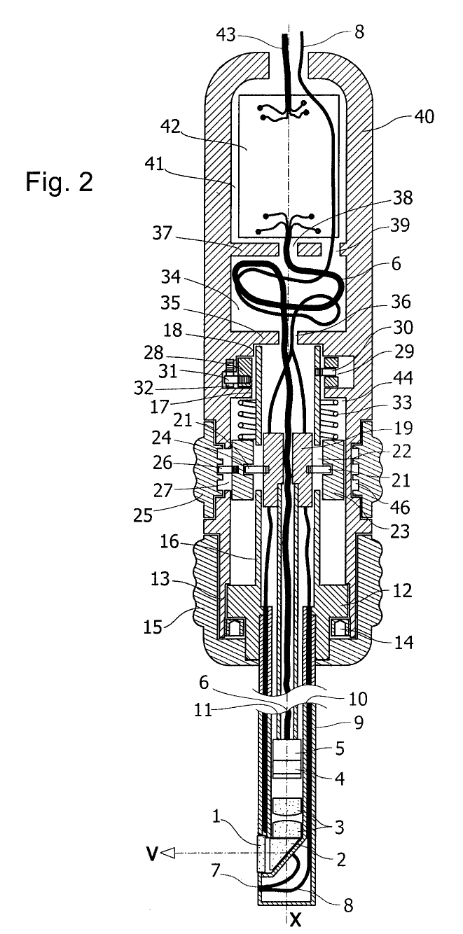

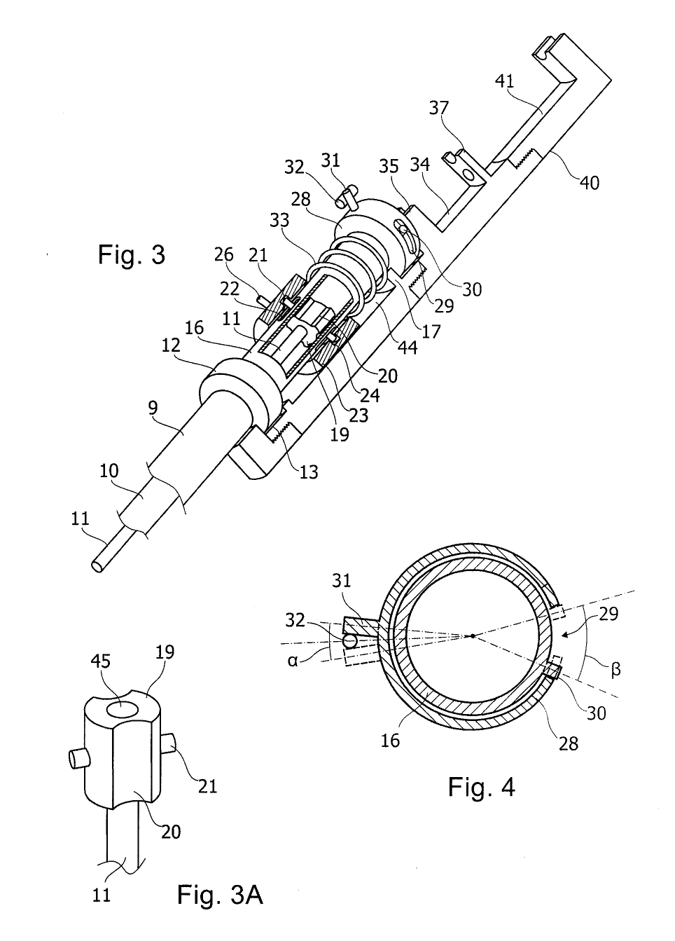

FIG. 2 shows the videoendoscopic probe. In FIG. 2, the probe comprises a focusing adjustment device and a rotation control device allowing the optical viewing axis V to rotate on substantially 360° around the longitudinal axis X of the inspection tube 9. FIG. 3 more particularly shows the focusing adjustment and rotation control devices and an area of the control handle 40.

In FIG. 2, the handle 40 has a cylindrical tubular shape comprising a distal cylindrical chamber 44, a central cylindrical chamber 34 separated from the chamber 44 by a wall 35, and a proximal cylindrical chamber 41 separated from the central chamber by a wall 37. The chamber 44 contains the rotation control and focusing adjustment mechanical devices.

The proximal chamber 41 contains a video processor 42. The proximal end of the chamber 41 comprises an orifice for letting a beam of lighting fibers 8 and a multicore cable 43, housed in the cable 50, pass. The multicore cable 43 allows the video processor 42 to be linked to the connector 52 provided to connect the video processor to auxiliary operation devices. The proximal end of the beam of lighting fibers 8 is attached to the lighting connector 51. The beam of lighting fibers 8 directly passes through the chamber 41 and an orifice 39 made in the wall 37, to reach the central chamber 34. The chamber 34 contains loops of a multicore electric cable 6 linking the video processor to a distal image sensor, as well as loops of the beam of lighting fibers 8, these loops being formed during the rotation of the inspection tube 9. The cable 6 passes through the wall 37 through a central orifice 38 and the wall 35 through a central orifice 36. The beam 8 passes through the wall 35 through the orifice 36.

A distal part of the distal chamber 44 is encircled by the rotation control ring 15 attached to the proximal end of the inspection tube 9. A central part of the chamber 44 is encircled by the focusing control ring 25.

In FIGS. 2 and 3, the inspection tube 9 houses an intermediate tube 10 attached to the tube 9 and a central tube 11 slidably, but not rotably housed in the tube 10. The distal end of the tube 10 houses an optoelectronic device comprising the optical window 1, a deviator prism 2 associated to an objective 3, and an image sensor 4 associated to an interface circuit 5. The window 1 is laterally arranged on a distal end of the tube 9. The prism 2 and the objective 3 are attached to the distal end of the tube 10. The image sensor 4 and the interface circuit 5 are attached to the distal end of the tube 11. The multicore electric cable 6 is housed in the tube 11 and connected to the interface circuit 5.

The beam of lighting fibers 8 is housed in an annular volume comprised between the tubes 9 and 10. The lighting window 7 at the distal end of the beam 8 is laterally arranged on the end of the tube 9, so as to form a lighting cone which axis is parallel to the optical viewing axis V.

The handle 40 houses a cylindrical tube 16 opened on both of its ends and attached to the proximal end of the inspection tube 9. The rotation around its axis of the inspection tube 9 therefore causes the rotation of the cylindrical tube 16, inside the handle 40. Bearings 13 and 17 are provided in the handle 40 for maintaining the tube 16 during its rotation movement.

The focusing control device comprises a cylindrical tubular central part 19 opened on both of its ends, a freely rotating ring 23, the central tube 11 and the control ring 25. The part 19, hereinafter referred to as “shuttle”, is attached to the proximal end of the central tube 11. The shuttle 19 is slidably but not rotatably housed in the tube 16. The shuttle 19 which is shown in greater details in FIG. 3A, comprises two external radial fingers 21, diametrically opposed, which are slidably fit into two diametrically opposed longitudinal slots 22 formed in the tube 16. The ends of the fingers 21 are slidably housed in an internal annular groove 24 formed in the freely rotating ring 23 encircling the tube 16. The tube 16 may thus freely rotate with the shuttle 19 in the ring 23, whatever the longitudinal position of the shuttle 19 inside the tube 16. The shuttle 19 comprises two external longitudinal bores 20, diametrically opposed, intended for letting pass the lighting fibers 8 and an axial cylindrical orifice 45 provided for letting pass the multicore electric cable 6.

The ring 23 comprises an external radial cylindrical finger 26 slidably moving in a longitudinal slot 27 made in the handle 40. The end of the finger 26 is housed in a helical internal thread 46 formed in the ring 25. Thus, the rotation of the ring 25 causes a longitudinal movement of the finger 26 and therefore of the ring 23 linked to the shuttle 19 by the fingers 21. As the shuttle 19 is attached to the tube 11, a longitudinal movement of the tube 11 causes an axial movement of the image sensor 4 associated to the interface circuit 5 in relation to the objective 3. Consequently, the ring 25 allows the sharpness of the video image supplied by the image sensor 4 to be adjusted. The helical thread 46 has a profile complementary to that of the finger 26. The length of the slot 22 (according to the axis X) defines the axial travel of the image sensor in relation to the objective 3.

A device for adjusting the longitudinal clearance may be provided to suppress an angular hysteresis defect of the focusing control ring 25 and resulting from longitudinal clearances of the focusing control device. To that end, the clearance adjustment device comprises a helical return spring 33 encircling the proximal end of the tube 16. The spring 33 is compressed between the distal face of the bearing 17 supporting the tube 16 and the proximal face of the freely rotating ring 23. Thus, the spring 33 allows the ring 23 and therefore the shuttle 19 to be pushed toward their extreme distal positions.

The rotation control device of the videoendoscopic probe around its longitudinal axis X, comprises the tube 16, a hub 12 attached to the distal end of the tube 16 and the rotation control ring 15 fixed to the hub. The tube 16 comprises an external radial cylindrical finger 30 cooperating with a stop formed by a longitudinal finger 32 attached to the handle 40.

The proximal end of the inspection tube 9 is rotatably housed in the distal part of the handle 40. The proximal end of the tube 9 is fixed to the hub 12 which encircles it. The hub 12 may freely rotate inside the bearing 13 made in the cylindrical distal part of the handle 40. A ring 14 comprising an external thread and encircling the central part of the hub 12 is screwed into the distal end of the handle 40, so as to maintain the hub in the handle.

The hub 12 is fixed in its distal part to the rotation control ring 15 and is attached in its proximal part to the distal end of the tube 16. The ring 15 therefore allows the cylindrical tube 16 to rotate inside the distal chamber 44. In one embodiment, the hub 12 and the tube 16 are made in a single part.

Thus, the rotation control device comprising the tube 16, linked to the hub 12 and the ring 15 and the tube 9 may substantially turn by an angle slightly inferior to 360°, the rotation of the whole being limited in both directions by the finger 32. The rotation of the tube 16 by the control ring 15 drives in rotation the tubes 9 and 10, as well as the shuttle 19 linked to the tube 16 by the fingers 21 which are only axially mobile in relation to the tube 16 due to the longitudinal direction of the slots 22. The result is that the tube 11 also turns substantially by the same angle as the tube 16. Consequently, the whole optoelectronic device also substantially turns by the same angle as the tube 16.

In addition, the mechanical link performed by the annular groove 24 between the freely rotating ring 23 and the shuttle 19, authorizes a free rotation of the tube 16 with the shuttle 19, controlled by the ring 15, without driving in rotation the focusing adjustment device comprising the ring 23, the control ring 25 and the tube 11. This mechanical link between the ring 25 and the shuttle 19 also authorizes an axial movement of the shuttle 19 inside the tube 16, whatever the angular position of the tube 16. Thus, there is no interaction between the rotation control device and the focusing adjustment device.

In one embodiment, a mobile stop device is provided for extending the rotation of the rotation control device beyond 360°. To that end, the mobile stop device comprises a freely rotating ring 28 encircling the tube 16 and comprising a radial slot in the shape of an arc of circle 29 in which the radial finger 30 attached to the tube 16 moves. The ring 28 is longitudinally maintained between the proximal face of the bearing 17 and the distal face of a bearing 18 attached to the wall 35 in the handle 40. The ring 28 comprises an external radial finger 31 coming to a stop onto the finger 32 attached to the handle 40. Thus, when the finger 31 stops against the finger 32 in a rotation direction or the other around the axis X, the tube 16 may go on turning thanks to the slot 29 in which the finger 30 moves, until the latter comes to a stop on one or the other end of the slot 29.

FIG. 4 shows the tube 16 and the ring 28. The ring 28 is shown in its extreme positions on each side of the finger 32, and the tube 16 is shown in its extreme positions in relation to the ring 28. The maximum rotation angle of the tube 16 is thus substantially equal to 360°−α+β, where α is the angular difference between the two extreme positions of the ring 28, and β is the angular difference between the two extreme angular positions of the tube 16 in relation to the ring 28. The angle α is substantially equal to the angular difference corresponding to the diameter of the finger 32, plus the angular difference corresponding the diameter of the finger 31. The angle β is substantially equal to the angle corresponding to the length of the slot 29 minus the angular difference corresponding the diameter of the finger 30. If the slot 29 has a length such that the angle β is superior to the angle α, the inspection tube 9 may turn by an angle superior to 360°.

Thanks to these measures, it is possible to perform a total panoramic observation of the observed area.

It will appear clearly to those skilled in the art that the present disclosure is susceptible of various embodiments. In particular, the disclosure is not limited to the embodiments previously described. Thus, it is within the reach of those skilled in the art to provide other modes of mechanical coupling or other modes of movement transmission of the various parts of the focusing and rotation control devices. For example, other coupling modes than fingers sliding in slots or grooves may be provided to mechanically couple two parts in translation or rotation.

In addition, the stop performed by the finger 32 is not necessary, but simply makes it possible to avoid the multicore cable 6 and the beam of fibers 8 from being subjected to too significant torsional stress, due to an excessive rotation of the inspection tube 9. The aim of providing the chamber 34 for receiving loops of the cable 6 and the beam of fibers 8 is also to limit stress, while authorizing a significant maximum rotation angle of the viewing axis V. Thus, the length of the loops in the housing 34 depends on the maximum rotation angle of the viewing axis V, which is not necessarily superior to 360°. In fact, given the angular width of the field of the objective 3, a rotation of less than 360° of the viewing axis may be sufficient to obtain a 360° panoramic observation.

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A deviated viewing rigid videoendoscopic probe with adjustable focusing, comprising:
a rigid inspection tube having a longitudinal axis and a distal part comprising an image capture device having a viewing axis different from the longitudinal axis; and
a control handle attached to the inspection tube and including a rotation control device for making the viewing axis turn around the longitudinal axis, and a focusing control device configured to control the image capture device for longitudinally moving in the inspection tube a part of the image capture device, the rotation control device and the focusing control device being respectively configured to make the viewing axis turn and focus the image capture device independently from one another.

2. The probe according to claim 1, wherein the image capture device comprises:
an objective;
an image sensor comprising a photosensitive surface on which forms an image supplied by the objective; and
a deviating optical element associated with the objective to laterally deviate an optical axis of the objective according to a viewing axis, the focusing control device being configured to axially move the image sensor in relation to the objective.

3. The probe according to claim 2, wherein the focusing control device comprises an axially mobile tube that includes a distal end attached to the image sensor and which axial movement is controlled by the focusing control device.

4. The probe according to claim 1, wherein the focusing control device comprises a control ring mounted mobile in rotation on the control handle, and mechanically coupled to a freely rotating ring so that a rotation of the control ring causes an axial translation of the freely rotating ring, the freely rotating ring being mechanically coupled in translation, but not in rotation, to a central part mechanically coupled to the image capture device.

5. The probe according to claim 4, wherein the focusing control device comprises an axial clearance device comprising an axial spring configured to return the freely rotating ring and the central part toward an extreme position.

6. The probe according to claim 1, wherein the rotation control device is configured to turn the inspection tube and the image capture device.

7. The probe according to claim 1, wherein the rotation control device comprises a control ring mounted mobile in rotation around the distal end of the control handle, and attached to the inspection tube and a cylindrical tube mobile in rotation but not in translation in the control handle, the cylindrical tube being mechanically coupled in rotation, but not in translation to a central part mechanically coupled to the image capture device.

8. The probe according to claim 1, wherein the rotation control device comprises a stop attached to the control handle and configured to limit rotation in both directions of the viewing axis.

9. The probe according to claim 1, wherein the rotation control device comprises a mobile stop device configured to extend the maximum rotation angle of the viewing angle to a value superior to 360°.

10. The probe according to claim 9, wherein the rotation control device comprises a control ring attached to the inspection tube and a cylindrical tube mobile in rotation in the control handle, the mobile stop device comprising a freely rotating ring mechanically coupled in rotation to the cylindrical tube by a finger moving in an annular slot made in the freely rotating ring, the freely rotating ring comprising a finger cooperating with a stop attached to the control handle.

11. The probe according to claim 1, comprising a beam of lighting fibers axially passing through the control handle and the inspection tube up to a lighting window arranged at the distal end of the inspection tube.

12. The probe according to claim 11, wherein the beam of lighting fibers passes through the inspection tube at an exterior of a focusing control tube, axially mobile in the inspection tube.

13. The probe according to claim 11, wherein the control handle comprises a chamber housing loops of the beam of lighting fibers and loops of a multicore cable linked to the image capture device.

14. The probe according to claim 1, comprising a video processor housed in a chamber of the control handle and linked to the image capture device by a multicore cable axially passing through the inspection tube.

15. The probe according to claim 14, wherein the multicore cable axially passes through the inspection tube in an axially mobile central tube in the inspection tube.
